(19) United States
(12) Patent Application Publication
(10) Pub. No.: US 2020/0352972 A1
(43) Pub. Date: Nov. 12, 2020

(54) ALDOXORUBICIN COMBINATION TREATMENTS AND METHODS

(71) Applicant: NANTCELL, INC., Culver City, CA (US)

(72) Inventors: Patrick SOON-SHIONG, Culver City, CA (US); John H. LEE, Culver City, CA (US); Shahrooz RABIZADEH, Culver City, CA (US); Kayvan NIAZI, Culver City, CA (US)

(21) Appl. No.: 16/640,336

(22) PCT Filed: Sep. 5, 2018
(86) PCT No.: PCT/US2018/049518

§ 371 Date: Feb. 19, 2020

Related U.S. Application Data
(60) Provisional application No. 62/554,742, filed on Sep. 6, 2017.

(51) Int. Cl. A61K 31/7056; A61K 47/64; A61K 35/17; A61K 38/20; A61P 35/00; A61K 39/00; A61K 39/12; A61K 39/02; A61K 33/243; A61K 31/7068; A61K 31/513; A61K 31/675; A61K 31/704; A61K 31/337; A61K 31/495; A61K 31/4995; A61K 38/08

(52) U.S. Cl. CPC A61K 31/7056; A61K 38/08; A61K 35/17; A61K 38/2013; A61K 38/2086; A61K 38/2046; A61K 38/20; A61P 35/00; A61K 39/0002; A61K 39/12; A61K 39/02; A61K 33/243; A61K 31/7068; A61K 31/513; A61K 31/675; A61K 31/704; A61K 31/337; A61K 31/495; A61K 31/4995; A61K 47/643

(57) ABSTRACT

Contemplated cancer therapies use aldoxorubicin as an immunomodulator of a tumor microenvironment to increase therapeutic effects of immune therapeutic compositions.

This application claims priority to US Provisional application with the Ser. No. 62/554,742, which was filed Sep. 6, 2017.

### FIELD OF THE INVENTION

The field of the invention is compositions and methods for cancer treatment, especially as it relates to immune therapeutic drugs in combination with targeted forms of doxorubicin.

### BACKGROUND

The following description includes information that may be useful in understanding the present invention. It is not an admission that any of the information provided herein is prior art or relevant to the presently claimed invention, or that any publication specifically or implicitly referenced is prior art.

Aldoxorubicin ((6-maleimidocaproyl) hydrazone of doxorubicin) is a prodrug form of doxorubicin that can be conjugated to thiol groups in various proteins, and especially to the thiol group of C34 in albumin when injected into an individual. Due to the acid labile nature of the hydrazine group, doxorubicin is hydrolytically cleaved from albumin once the doxorubicin-albumin conjugate encounters an acidic milieu as is often found in the cancer microenvironment. Therefore, aldoxorubicin is expected to specifically release free doxorubicin in the tumor microenvironment. Advantageously, circulating albumin also tends to preferentially accumulate in tumors, most likely due to gp60-mediated transcytosis through the endothelium of the tumor neovasculature. Consequently, it is thought that aldoxorubicin presents an attractive therapeutic modality to specifically target the tumor microenvironment and to exert its pharmaceutical effect on DNA topoisomerase II to so disrupt rapidly dividing cancer cells.

To that end, various clinical trials have been undertaken, including second-line treatment for glioblastoma (clinical trial identifier NCT02014844), treatment for Kaposi's sarcoma (clinical trial identifier 2029430), advanced or metastatic pancreatic ductal adenocarcinoma (clinical trial identifier NCT01580397), and metastatic small cell lung cancer (clinical trial identifier NCT02200757). Aldoxorubicin has also been reported in a combination with ifosfamide for treatment of metastatic or locally advanced sarcoma (clinical trial identifier NCT02235701). Notably, aldoxorubicin has not been used in combination with immune therapeutic agents, presumably due to suspected adverse effects from DNA damage response, and epigenetic and transcriptomic deregulation in various cells exposed to doxorubicin. Moreover, doxorubicin has also been reported as an immune suppressant (see e.g., Ann Plast Surg. 2012 February; 68(2):215-21).

All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Thus, even though limited combinations of aldoxorubicin in the treatment of cancer are known in the art, there is still a need to provide improved combination therapies, particularly in combination with immune therapeutic compositions.

### SUMMARY OF THE INVENTION

The inventive subject matter provides various compositions and methods of treatment of cancer in which aldoxorubicin is co-administered with an immune therapeutic composition that typically includes a vaccine component and/or a cell-based component, and that is administered under a temporo-spatial treatment regimen to reverse the escape phase of cancer immune editing and help establish the equilibrium and/or elimination phase of cancer immune editing.

In one aspect of the inventive subject matter, the inventors contemplate a method of treating a tumor that includes a step of treating tumor cells within an acidic and hypoxic tumor microenvironment with at least a first pharmaceutical composition that reduces immune suppression in the tumor microenvironment to thereby revert an escape phase of the tumor cells. In another step, the tumor cells are treated with an immune therapeutic composition that comprises a vaccine component and a cell-based component to thereby induce an elimination phase of the tumor cells. Where desired, contemplated methods may further comprise a further step of maintaining an equilibrium phase of the tumor cells by administering at least a second pharmaceutical composition that biases an immune response towards a T_{H}1 response.

The first pharmaceutical composition preferably comprises a drug that binds to a thiol group of an albumin or a drug that is bound to an albumin, wherein the albumin is optionally a nanoparticulate albumin, and especially preferred drugs include aldoxorubicin. In other aspects, the drug may also include Bendamustine, Bortezomib, Cabazitaxel, Chlorambucil, Cisplatin, Cyclophosphamide, Dasatinib, Docetaxel, Doxorubicin, Epirubicin, Erlotinib, Etoposide, Everolimus, Gefitinib, Idarubicin, Hydroxyurea, Imatinib, Lapatinib, Melphalan, Mitoxantrone, Nilotinib, Oxiplatin, Paclitaxel, Pazopanib, Pemetrexed, Rapamycin, Romidepsin, Sorafenib, Vemurafenib, Sunitinib, Teniposide, Vinblastine, Vinorelbine, or Vincristine, and/or an antibody or fragment thereof may be bound to the albumin. Therefore, and viewed from a different perspective, the first pharmaceutical composition may comprises a drug that inhibits at least one of a T-reg cell, a myeloid derived suppressor cell, and a M2 macrophage, and especially suitable drugs include cisplatin, gemcitabine, 5-fluorouracil, cyclophosphamide, doxorubicin, temozolomide, docetaxel, paclitaxel, trabectedin, and RP-182. Additionally, the first pharmaceutical composition may also comprise a vascular permeability enhancer (e.g., a portion of IL2).

Suitable vaccine components may comprise a recombinant bacterial vaccine, a recombinant viral vaccine, or a recombinant yeast vaccine, typically genetically engineered to express a cancer associated antigen, a cancer specific antigen, and/or a patient- and tumor-specific neoepitope. For example, suitable cancer associated antigen include MUC1, CEA, HER2, Brachyury, and an oncogenic Ras mutant protein. While the cell-based component may comprises numerous cytotoxic cells, it is generally preferred that the cell-based component comprises a natural killer cell, and especially an aNK cell, a haNK cell, or a taNK cell.

Moreover, contemplated methods may also include an additional step of administering an immune stimulatory cytokine (e.g., IL-2, IL-7, IL-15, IL-17, IL-21, an IL-15 superagonist), a checkpoint inhibitor (e.g., PD-1 inhibitor or CTLA4 inhibitor), and/or a step of administering low dose radiation to the tumor.

Therefore, and in yet another aspect of the inventive subject matter, the inventors also contemplate uses of aldoxorubicin and a method of immunomodulation of a tumor microenvironment that includes a step of administering aldoxorubicin to the tumor microenvironment in an amount effective to immunomodulate the tumor microenvironment.

Most typically, the tumor microenvironment is hypoxic and/or acidic. With respect to the immunomodulation it is contemplated that the immunomodulation is a reduction or elimination of MDSC and/or M2 macrophages in the tumor microenvironment, an increased expression of a CD40 ligand and/or 4-1BB, and/or a Stat1-dependent antitumor immune response in the tumor microenvironment.

Various objects, features, aspects and advantages of the inventive subject matter will become more apparent from the following detailed description of preferred embodiments, along with the accompanying drawing.

### BRIEF DESCRIPTION OF THE DRAWING

FIG. 1 is a schematic overview of a treatment regimen according to the inventive subject matter for treatment of metastatic pancreatic cancer.

FIG. 2 depicts selected treatment trials and modalities for the treatment of FIG. 1.

FIG. 3 depicts exemplary results for one patient subject to the treatment of FIG. 1 (3.070).

FIG. 4 depicts exemplary results for another patient subject to the treatment of FIG. 1 (3.070).

FIG. 5 depicts exemplary results for a further patient subject to the treatment of FIG. 1 (3.070).

FIG. 6 depicts exemplary results for yet another patient subject to the treatment of FIG. 1 (3.070)

FIG. 7 is a schematic overview of a treatment regimen according to the inventive subject matter for treatment of metastatic triple negative breast cancer (TNBC).

FIG. 8 depicts a response summary for the treatment of FIG. 7.

FIG. 9 depicts exemplary results for one patient subject to the treatment of FIG. 7.

FIG. 10 is a schematic overview of a treatment regimen according to the inventive subject matter for treatment of metastatic squamous cell carcinoma.

FIG. 11 depicts a selected treatment trial and modalities for the treatment of FIG. 10.

FIG. 12 depicts exemplary results for one patient subject to the treatment of FIG. 10.

FIG. 13 depicts exemplary results for another patient subject to the treatment of FIG. 10.

FIG. 14 depicts exemplary results for a further patient subject to the treatment of FIG. 10.

FIG. 15 depicts exemplary result summaries for selected treatments contemplated herein.

### DETAILED DESCRIPTION

The inventors have now discovered various compositions and methods of treatment of cancer in which aldoxorubicin is co-administered with an immune therapeutic composition that includes a vaccine component and/or a cell-based component. More specifically, aldoxorubicin may provide at least two distinct advantages in immunotherapy that are different from the known effects of doxorubicin on DNA topoisomerase II.

First, delivery of doxorubicin is preferential into the acidic tumor microenvironment via acid catalyzed hydrolysis of aldoxorubicin, and second, inhibition of MDSC/M2 macrophages by the so delivered doxorubicin in the tumor microenvironment. In addition, Doxorubicin was also reported to enhance CD4⁺ T-cell immune responses by inducing expression of CD40 ligands and 4-1BB (Int Immunopharmacol. 2009; 9:1530-9), and was shown to enhance the Stat1-dependent antitumor immune response (Eur J Immunol. 2013; 43:2718-29). Advantageously, aldoxorubicin can perform such functions in the acidic and hypoxic microenvironment and is therefore thought to counteract the immune suppressive nature of the acidic and hypoxic microenvironment. These functions and the specificity to the tumor microenvironment are particularly beneficial where the cancer treatment is a temporo-spatial treatment. Viewed from yet another perspective, it should be recognized that aldoxorubicin is used as an immunomodulatory agent that is specific to the hypoxic and acidic tumor microenvironment.

Therefore, and in one aspect of the inventive subject matter, compositions and methods for cancer therapy are presented to maximize immunogenic cell death (ICD) while maintaining and augmenting the patients' antitumor adaptive and innate responses to cancers. To that end, the treatment methods and uses of specific compounds and compositions presented herein take in at least some cases advantage of lower, metronomic doses of both cytotoxic chemotherapy and radiation therapy to so induce damage associated molecular patterns (DAMP) signals and tumor cell death while minimizing suppression of the immune system. In addition, contemplated methods also include use of various immunomodulatory agents, vaccines, checkpoint inhibitors, cell-based compositions, and fusion proteins to augment and stimulate the patient's adaptive and innate immune responses.

Notably, by overcoming immunosuppressed tumor microenvironment by aldoxorubicin and other drugs as presented below, the elimination phase of cancer can be reinstated through effector cells (e.g., mature dendritic cells, NK cells, cytotoxic T-cells, memory T-NK cells), that are preferably activated by combination therapy using fusion proteins, adenovirus and yeast vector vaccines, and/or natural killer cells. It should also be appreciated that such combinations may be targeted to mutational patterns that are specific to the patients (e.g., via targeting patient- and tumor-specific neoepitopes). Therefore, and among other benefits, the risk of off-target stimulation of an immune response is significantly reduced.

Most preferably, contemplated compounds and compositions will be administered in a temporo-spatial orchestration of a combination of various immunotherapeutic products to so immunomodulate the tumor microenvironment, activate the innate adaptive immune system, and to induce immunogenic cell death (ICD). More specifically, the inventors contemplate that such approach will result in coordinated effects, and especially in:

(1) Breaking the escape phase of cancer immune editing, preferably by overcoming the tumor immunosuppressed state. In addition to administration of aldoxorubicin, such treatment is preferably informed by tissue and/or liquid biopsies, and preferably performed using low-dose metronomic chemotherapeutic agents that are capable of inhibiting T-Reg, MDSCs, and M2 Macrophages, and/or by inhibition or blocking action of cytokines (e.g., TGF β, IL-6, IL-8) that enhance immunosuppression;

(2) Inducing the elimination phase of cancer immune editing, preferably done by up-regulating and/or induction of damaged associated molecular patterns (DAMP) signals, up-regulating of tumor associated MHC restricted antigens and stress receptors (NKG2D), up-regulating tumor specific receptors such as PD-L1 and/or via low-dose radiation, administration of immunomodulatory drugs (IMiDs) and histone deacetylase (HDAC) agents, and/or activation of dendritic cells, natural killer cells, cytotoxic T-cells, memory T and/or Natural Killer (NK) cells through adenovirus, bacterial, and/or yeast vector vaccines, cytokine fusion protein administration, checkpoint inhibitors, and/or NK cell therapy infusion; and

(3) Reinstatement of the equilibrium phase of cancer immune editing, which can be achieved by maintaining T_{H}1 status of the patient's immune system with vaccine boosters, cytokine fusion protein maintenance, and/or regular exogenous NK infusions.

To that end, and among other contemplated options, preferred treatment components include (a) albumin bound chemotherapy combinations (especially including albumin bound aldoxorubicin) to enter the tumor microenvironment to overcome the suppressive environment in the tumor, (b) antigen producing vaccine entities (e.g., recombinant adenovirus, bacteria, and/or yeast) that directly or indirectly deliver tumor associated antigens and/or patient- and tumor-specific neoepitopes to immune competent cells to activate immature dendritic cells (e.g., in a patient and tumor specific manner using neoepitopes or general manner using cancer associated antigens) to induce and/or enhance an adaptive immune response, (c) natural killer cells, which may be endogenous (e.g., by stimulation with IL-15 or IL-15 superagonist) and/or exogenous (e.g., genetically modified NK cells such as aNK, haNK, taNK cells) to induce and/or enhance an innate immune response, and (d) endogenous activated memory T- and/or NK-cells to sustain long term remission, preferably activated via vaccine, cell therapy, and fusion proteins where desired (e.g., genetically engineered fusion protein cytokine stimulators and/or checkpoint inhibitors).

Therefore, it should be appreciated that the tumor microenvironment can be modulated with aldoxorubicin to initiate a break in the escape phase of tumor immune editing in a specific manner in which aldoxorubicin is delivered to the tumor microenvironment using transcytosis (gp60-mediated) of albumin to which the aldoxorubicin is bound. Once the albumin conjugates are in the tumor microenvironment, doxorubicin is released and reduces MDSCs and M2 macrophages, which are significant contributors to immune suppression.

In this context, it should be noted that aldoxorubicin is not employed in its previously known function as a DNA topoisomerase II inhibitor, but as an agent to immunomodulate the hypoxic and acidic tumor microenvironment. Such use is particularly desirable as vaccine- and cell-based immunotherapeutics may otherwise be substantially less effective when exposed to the hypoxic environment of the tumor.

Of course, it should be appreciated that while aldoxorubicin is a preferred agent to reduce or eliminate immune suppression in a tumor microenvironment, various other drugs may also be employed (in addition or in the alternative), including cytoxan, 5-fluorouracil, leucovorin, and/or bevacizumab using dosages and treatment regimens well known in the art. Aldoxorubicin will typically be administered in a dosage of between about 1 mg/m² to 500 mg/m², and more typically between 10 mg/m² to 100 mg/m², and most typically between 20 mg/m² to 80 mg/m². Thus suitable aldoxorubicin dosages will be 10-20 mg/m², 20-30 mg/m², 30-60 mg/m², 50-80 mg/m², or 60-100 mg/m². Regardless of the particular dose chosen, the biological effect of reduced immune suppression may be monitored by various manners, including tumor biopsies and immune cell analysis, circulating immune cell analysis, and/or analysis of circulating free nucleic acids from one or more specific immune cell type.

Immune therapy will preferably include at least a vaccine component and a cell-based component. Among other suitable options, it is typically preferred that the immune therapeutic composition is a cancer vaccine that is based on at least one of a bacterial vaccine, a yeast vaccine, and an (adeno)viral vaccine as described in more detail below. It should be appreciated that the cancer vaccines are preferably recombinant entities that have expressed in the intracellular space one or more tumor associated antigens and/or tumor neoepitopes, or that the recombinant entity is a recombinant viral expression vector that encodes one or more tumor associated antigens and/or tumor neoepitope. In further preferred aspects, it should also be noted that the vaccine compositions may be administered sequentially (e.g., first bacterial, then yeast, then viral), or that only one or two vaccine compositions are used (e.g., only adenoviral or bacterial vaccine). Of course, it should be appreciated that the recombinant protein(s) or nucleic acid(s) encoding the protein(s) may be the same in all vaccine compositions, overlapping, or different.

With respect to the enhancement of the innate immune response in the elimination phase it is generally preferred that the innate immune response may be from the patient's own immune system or via exogenous immune competent cells. For example, where the patient's innate immune response is enhanced, proliferation and activity of natural killer cells and activated T-cells may be boosted using one or more immune stimulatory cytokines as discussed in more detail below. Alternatively, or additionally, the patient may also receive allogenic NK cells, and most preferably activated NK cells (such as aNK cells, haNK cells, or taNK cells) and/or recombinant T-cells with a chimeric T cell receptor. NK transfusion, and especially aNK and haNK transfusion advantageously amplify prior stress signals present on the tumor cells in the TME (typically induced by metronomic low dose chemo therapy, low dose radiation, and/or endocrine deprivation). Additionally, haNK cells may be coupled via the high affinity CD16 receptor to one or more antibodies that bind tumor associated antigens or neoepitopes. As such, the innate immune response may be specifically directed to a tumor cell. The elimination phase may be further enhanced or supported by administration of one or more cytokines, fusion proteins, and/or chemokines as is further discussed in more detail below.

For example, recombinant yeast and viruses are especially deemed suitable, and recombinant adenoviral systems (such as Ad5 type) with reduced antigenicity are described in WO 2017/143092, WO 2018/005973, WO 2017/161360, and WO 2016/164833 (and their corresponding national phase publications). Such viruses can, for example, be prepared in a method that includes one step of identifying a cancer-related neoepitope of a patient, a further step of determining binding of the neoepitope to an HLA-type of the patient, and determining an expression level of the neoepitope, a still further step of selecting at least one co-stimulatory molecule, and a step of genetically modifying a virus to include a nucleic acid encoding the at least one co-stimulatory molecule and the cancer-related neoepitope. With respect to the virus, it is generally referred that the virus is an adenovirus or a replication deficient virus. Moreover, it is further preferred that the virus is non-immunogenic. Thus, especially preferred viruses include an adenovirus, and especially an Ad5 [E1⁻E2b⁻].

Where cancer-related neoepitopes of the patient are employed as antigens it is contemplated that such (neo)antigens are preferably identified in silico by location-guided synchronous alignment of omics data of tumor and matched normal samples, and contemplated methods may further comprise a step of predicting the HLA type of the patient in silico. Consequently, HLA matched epitopes are especially preferred. While not limiting to the inventive subject matter, it is preferred that the expression level of the neoepitope is at least 20% compared to a matched normal sample.

It is further contemplated that the recombinant entity (e.g., bacterium, yeast, virus) may also include one or more sequences that encode one or more co-stimulatory molecule, including selected from the group of B7.1 (CD80), B7.2 (CD86), CD30L, CD40, CD40L, CD48, CD70, CD112, CD155, ICOS-L, 4-1BB, GITR-L, LIGHT, TIM3, TIM4, ICAM-1, and LFA3 (CD58). Moreover, the nucleic acid may further include a sequence encoding a cytokine (e.g., IL-2, IL-7, IL-12, IL-15, an IL-15 superagonist (IL-15N72D), and/or an IL-15 superagonist/IL-15RαSushi-Fc fusion complex). Alternatively, or additionally, the nucleic acid further may also include a sequence encoding at least one component of a SMAC (e.g., CD2, CD4, CD8, CD28, Lck, Fyn, LFA-1, CD43, and/or CD45 or their respective binding counterparts). Where desired, the nucleic acid may additionally comprise a sequence encoding an activator of a STING pathway, such as a chimeric protein in which a transmembrane domain of LMP1 of EBV is fused to a signaling domain of IPS-1. Such modifications are thought to even further enhance development of an adaptive immune response by providing additional signals for activation of the adaptive immune response.

With respect to the cell based component of the immune therapeutic composition it is contemplated that the cells are NK cells, T cells, and recombinant versions thereof. For example, in one particularly preferred aspect of the inventive subject matter, the NK cell is a NK-92 derivative and is preferably genetically modified to have a reduced or abolished expression of at least one killer cell immunoglobulin-like receptor (KR), which will render such cells constitutively activated (via lack of or reduced inhibition). Therefore, suitable modified cells may have one or more modified killer cell immunoglobulin-like receptors that are mutated such as to reduce or abolish interaction with MHC class I molecules. Of course, it should be noted that one or more KIRs may also be deleted or expression may be suppressed (e.g., via miRNA, siRNA, etc.). Most typically, more than one KIR will be mutated, deleted, or silenced, and especially contemplated KIR include those with two or three domains, with short or long cytoplasmic tail. Viewed from a different perspective, modified, silenced, or deleted KIRs will include KIR2DL1, KIR2DL2, KIR2DL3, KIR2DL4, KIR2DL5A, KIR2DL5B, KIR2DS1, KIR2DS2, KIR2DS3, KIR2DS4, KIR2DS5, KIR3DL1, KIR3DL2, KIR3DL3, and KIR3DS1. Such modified cells may be prepared using protocols well known in the art. Alternatively, such cells may also be commercially obtained from NantKwest (see URL www.nantkwest.com) as aNK cells (‘activated natural killer cells).

In another example, the genetically engineered NK cell may also be an NK-92 derivative that is modified to express the high-affinity Fcγ receptor (CD16). Sequences for high-affinity variants of the Fcγ receptor are well known in the art, and all manners of generating and expression are deemed suitable for use herein. Expression of such receptor is believed to allow specific targeting of tumor cells using antibodies that are specific to a patient's tumor cells (e.g., neoepitopes), a particular tumor type (e.g., her2neu, PSA, PSMA, etc.), or that are associated with cancer (e.g., CEA-CAM). Advantageously, such antibodies are commercially available and can be used in conjunction with the cells (e.g., bound to the Fcγ receptor). Alternatively, such cells may also be commercially obtained from NantKwest as haNK cells (‘high-affinity natural killer cells).

In yet a further aspect of the inventive subject matter, the genetically engineered NK cell may also be genetically engineered to express a chimeric T-cell receptor. In especially preferred aspects, the chimeric T-cell receptor will have a scFv portion or other ectodomain with binding specificity against a tumor associated antigen, a tumor specific antigen, and a cancer neoepitope. As noted before, there are numerous manners of genetically engineering an NK cell to express such chimeric T-cell receptor, and all manners are deemed suitable for use herein. Alternatively, such cells may also be commercially obtained from NantKwest as taNK cells (‘target-activated natural killer cells’).

Likewise, where T cells are used as part of the immune therapeutic composition, it is generally preferred that the T cell is an autologous T cell, which may have been ex vivo expanded or (re)activated, possibly in the presence of a patient specific (neo)antigen. Alternatively, the T cell may also be a CAR-T cell expressing a chimeric antigen receptor, typically having an ectodomain that has affinity to a patient and tumor specific antigen.

In still further contemplated aspects, it should be appreciated that one or more cytokines or cytokine analogs may be administered that support immune function, and especially expansion of activated T cells and K cells. Therefore, especially preferred cytokines and analogs include IL-2, IL-15, and IL-21, and particularly ALT-803 (see e.g., Cytokine 2011; 56(3):804-10)) and T×M constructs having an IL-15 agonist and receptor portion (see e.g., URL: altorbioscience.com/our-science/il-15-protein-superagonist-and-scaffold-technology/#T×M). Such stimulation is contemplated to assist in T memory cell formation, and especially in T_{SCM} cell formation.

### Examples

Combination Immunotherapy in Subjects with Multiple Myeloma:

Therapeutic compositions and modalities used include various biological molecules and compositions as shown in Table 1 below.

TABLE 1

| | |
|---|---|
| ALT-803 | Recombinant human super agonist interleukin-15 (IL-15) complex, |
| | IL-15N72D: IL-15RαSu/IgG1 Fc complex (Altor Bioscience Corp., |
| | 2810 N Commerce Pkwy, Miramar, FL 33025) |
| ETBX-061 | Recombinant Adenovirus (Ad5 [E1-, E2b-]-MUC1) that encodes |
| | MUC1 for expression of MUC1 in infected cells |
| GI-4000 | Heat-killed S. cerevisiae yeast expressing the mutated RAS oncoproteins |
| NK-92 [CD16.158V, | NK92 derivative cells with high affinity CD16 variant and recombinant |
| ER IL-2] | intracellular expression of IL-2 (high-affinity activated Natural Killer |
| | cells, [haNK ™], NantKwest, 9920 Jefferson Blvd. Culver City, CA 90232) |
| Aldoxorubicin | Doxorubicin derivative ((6-maleimidocaproyl) hydrazone of doxorubicin). |
| Bortezomib | Velcade ([(1R)-3-methyl-1-({(2S)-3-phenyl-2-[(pyrazin-2-ylcarbonyl)- |
| | amino]propanoyl}amino)butyl]boronic acid) |
| Lenalidomide | Revlimid ((RS)-3-(4-Amino-1-oxo-1,3-dihydro-2H-isoindol-2-yl) |
| | piperidine-2,6-dione) |
| Elotuzumab | Implicity (humanized immunostimulatory antibody targeting CD319) |
| Dexamethasone | 1-dehydro-9a-fluoro-16a-methylhydrocortisone |
| Avelumab | Bavencio (Fully human anti-PD-L1 IgG1 lambda monoclonal antibody) |
| Cyclophosphamide | 2-[bis(2-chloroethyl)amino]tetrahydro-2H-1,3,2-oxazaphosphorine |
| | 2-oxide monohydrate |
| Omega-3-acid ethyl | Lovaza (Omega-3-acid ethyl esters) |
| esters | |
| Radiation | Stereotactic Body Radiation Therapy (SBRT); 8 Gy maximum |
| | (exact dose to be determined by the radiation oncologist) |

Treatment will be administered in 2 phases, an induction phase and a maintenance phase, as described below. Subjects will continue induction treatment for 6 cycles. After 6 treatment cycles, subjects will undergo CT or MRI to determine CR, PR, and PD rates. Those who have a pCR at the locoregional site and CR of metastatic disease will enter the maintenance phase. Subjects who do not have a pCR of locoregional disease will continue on 3 more cycles of neoadjuvant therapy (without SBRT) and then enter the maintenance phase. Subjects may remain on the maintenance phase for up to 1 year. Treatment will continue in the maintenance phase until the subject experiences PD or unacceptable toxicity (not correctable with dose reduction).

Tumor biopsies and exploratory tumor molecular profiling will be conducted at screening, at the end of the initial induction phase (18 weeks after the start of treatment), and during the maintenance phase (depending on response). Separate blood tubes will be collected every 4 weeks in the induction phase and every 8 weeks in the maintenance phase during routine blood draws for exploratory immunology and ctDNA/ctRNA analyses.

Tumors will be assessed at screening, and tumor response will be assessed every 8 weeks during the induction phase and every 12 weeks during the maintenance phase by computed tomography (CT), magnetic resonance imaging (MRI), or positron emission tomography-computed tomography (PET CT) of target and non-target lesions in accordance with Response Evaluation Criteria in Solid Tumors (RECIST) Version 1.1 and immune-related response criteria (irRC).

Prospective Tumor Molecular Profiling: Prospective tumor molecular profiling will be conducted to inform RAS mutational status and will be used to determine whether GI-4000 will be administered. All subjects will receive all other agents regardless of their tumor molecular profile. Prospective tumor molecular profiling will be performed on FFPE tumor tissue and whole blood (subject-matched normal comparator against the tumor tissue) collected at screening. Subjects will receive GI-4000 if their tumor is positive for specific RAS mutations, as determined by whole genome sequencing. GI-4000 is 4 separate products from the GI-4000 series (GI-4014, GI-4015, GI-4016, and GI-4020); each of these expresses a combination of mutated RAS oncoproteins. The specific RAS mutation will determine which GI-4000 product will be used for treatment (GI-4014 for G12V, GI-4015 for G12C, GI-4016 for G12D, GI-4020 for G12R or Q61H, and GI-4014, GI-4015, or GI-4016 for Q61L or Q61R).

Induction Phase: The induction phase comprises repeated 3 week cycles. The treatment regimen of ALT-803, Ad5 based MUC1 vaccine (ETBX-061,), yeast-based KRAS vaccine (GI-4000), haNK cells, aldoxorubicin, avelumab, cyclophosphamide, bortezomib, lenalidomide, dexamethasone, and omega-3-acid ethyl esters will be repeated every 3 weeks. Concurrent SBRT will be given during the first four cycles. Radiation will be administered to no more than 5 feasible tumor sites using SBRT. The induction phase will be conducted in accordance with the following dosing regimen:

Daily: Omega-3-acid ethyl esters (by mouth [PO] twice a day [BID] [3×1 g capsules and 2×1 g capsules]); Aspirin (81 mg).

Day 1, 8, every 3 weeks: Aldoxorubicin (40 mg/m²).

Days 1-14, every 3 weeks: Lenalidomide (25 mg daily tablet).

Days 1-5, 8-12, 15-19, every three weeks: Cyclophosphamide (50 mg PO BID).

Day 1, 8, 15, every three weeks: Dexamethasone (10 mg IV).

Days 1, 4, 8, 11, every three weeks: Bortezomib (1.0 mg/m² IV)

Day 5 (every 3 weeks for 3 doses then every 8 weeks thereafter): ETBX-061, (5×10¹¹ virus particles [VP]/vaccine/dose subcutaneously [SC]); GI-4000 (40 yeast units [YU]/vaccine/dose SC), 2 hours after administration of Ad5-based vaccines. Prospective tumor molecular profiling will determine whether GI-4000 will be administered, as described above.

Day 8 and 15, every 3 weeks: Elotuzumab (10 mg/kg, IV).

Day 8, 15 (during the first 2 cycles for a total of 4 doses): SBRT (not to exceed 8 Gy, exact dose to be determined by the radiation oncologist).

Day 9, and 16 every 3 weeks: ALT-803 (10 μg/kg SC 30 minutes prior to haNK infusion).

Day 9, 11, 16, and 18 every 3 weeks: haNK (2×10⁹ cells/dose IV).

Maintenance Phase:

The duration of the maintenance phase will be up to 1 year following completion of the last treatment in the induction phase. The maintenance phase will include repeated 3-week cycles. The treatment regimen of ALT-803, Ad5 based MUC1 vaccine (ETBX-061), yeast-based KRAS vaccine (GI-4000), haNK cells, aldoxorubicin, avelumab, cyclophosphamide, bortezomib, lenalidomide, dexamethasone, and omega-3-acid ethyl esters will be repeated every 3 weeks. The maintenance phase will be conducted in accordance with the following dosing regimen:

Daily: Omega-3-acid ethyl esters (by mouth [PO] twice a day [BID] [3×1 g capsules and 2×1 g capsules]); Aspirin (81 mg).

Day 1, every 3 weeks: Aldoxorubicin (20 mg/m²); Elotuzumab (10 mg/kg, IV); Dexamethasone (10 mg IV).

Days 1-14, every 3 weeks: Lenalidomide (15 mg daily tablet).

Days 1-5,8-12, 15-19, every 3 weeks: Cyclophosphamide (50 mg PO BID).

Days 1, 8, every three weeks: Bortezomib (0.7 mg/m² IV).

Day 2, every 3 weeks: ALT-803 (10 μg/kg SC 30 minutes prior to haNK infusion); haNK (2×10⁹ cells/dose IV).

Day 5 (every 3 weeks for 3 doses then every 8 weeks thereafter): ETBX-061, (5×1 virus particles[VP]/vaccine/dose subcutaneously [SC]); GI-4000 (40 yeast units [YU]/vaccine/dose SC), 2 hours after administration of Ad5-based vaccines. Prospective tumor molecular profiling will determine whether G-4000 will be administered, as described above.

Combination Immunotherapy in Subjects with TNBC:

Therapeutic compositions and modalities used include various biological molecules and compositions as shown in Table 2 below.

TABLE 2

| | |
|---|---|
| ALT-803 | Recombinant human super agonist interleukin-15 (IL-15) complex, |
| | IL-15N72D: IL-15RαSu/IgG1 Fc complex (Altor Bioscience Corp., |
| | 2810 N Commerce Pkwy, Miramar, FL 33025); |
| ETBX-011 | Recombinant Adenovirus (Ad5 [E1-, E2b-]-CEA) that encodes |
| | CEA for expression of CEA in infected cells; |
| ETBX-051 | Recombinant Adenovirus (Ad5 [E1-, E2b-]-Brachyury) that encodes |
| | Brachyury for expression of Brachyury in infected cells; |
| ETBX-061 | Recombinant Adenovirus (Ad5 [E1-, E2b-]-MUC1) that encodes |
| | MUC1 for expression of MUC1 in infected cells; |
| GI-6207 | Heat-killed S. cerevisiae yeast expressing CEA |
| GI-6301 | Heat-killed S. cerevisiae yeast expressing the human Brachyury |
| | (hBrachyury) oncoprotein |
| NK-92[CD16.158V, | NK92 derivative cells with high affinity CD16 variant and recombinant |
| ER IL-2] | intracellular expression of IL-2 (high-affinity activated Natural Killer |
| | cells, [haNK ™], NantKwest, 9920 Jefferson Blvd. Culver City, CA 90232) |
| Aldoxorubicin | Doxorubicin derivative ((6-maleimidocaproyl) hydrazone of doxorubicin). |
| Aspirin | Acetylsalicylic acid |
| Avelumab | Bavencio (Fully human anti-PD-L1 IgG1 lambda monoclonal antibody) |
| Cyclophosphamide | 2-[bis(2-chloroethyl)amino]tetrahydro-2H-1,3,2-oxazaphosphorine |
| | 2-oxide monohydrate |
| Paclitaxel | 5β,20-Epoxy-1,2α,4,7β,10β,13α-hexahydroxytax-11-en-9-one |
| | 4,10-diacetate 2-benzoate 13 ester with (2R,3S)-N-benzoyl-3-phenylisoserine |
| Omega-3-acid ethyl | Lovaza (Omega-3-acid ethyl esters) |
| esters | |
| Radiation | Stereotactic Body Radiation Therapy (SBRT); 8 Gy maximum |
| | (exact dose to be determined by the radiation oncologist) |

Treatment will be administered in 2 phases, a neoadjuvant phase and a post-operative phase, as described below. Subjects will receive the neoadjuvant phase treatment for 6 cycles. After 6 cycles, subjects will undergo CT or MRI to determine their current response status (ie, CR, PR, SD, or PD). Subjects will then undergo appropriate breast surgery and node dissection after which pCR will be evaluated. pCR will be defined as the absence of residual invasive cancer on hematoxylin and eosin evaluation of the complete resected breast specimen and all sampled regional lymph nodes following completion of neoadjuvant systemic therapy. Subjects will then enter the post-operative phase where they may remain for up to 6 weeks. Treatment will continue in the post-operative phase unless they experience unacceptable toxicity. The maximum time on treatment is 18 weeks in the neoadjuvant phase and 6 weeks in the maintenance phase.

Tumor biopsies and exploratory tumor molecular profiling will be conducted at screening, at the end of the neoadjuvant phase (18 weeks after the start of treatment), and during the post-operative phase. Separate blood tubes will be collected every 4 weeks in the neoadjuvant phase and every 8 weeks in the post-operative phase during routine blood draws for exploratory immunology and ctDNA/ctRNA analyses. Tumors will be assessed at screening, and tumor response will be assessed every 8 weeks during the neoadjuvant phase and every 12 weeks during the post-operative phase by computed tomography (CT), magnetic resonance imaging (MRI) of target and non-target lesions in accordance with Response Evaluation Criteria in Solid Tumors (RECIST) Version 1.1 and immune-related response criteria (irRC).

Neoadjuvant Phase:

The neoadjuvant phase will include 6 cycles. Each cycle is 3 weeks. The treatment regimen of ALT-803, Ad5 based vaccines (ETBX-011, ETBX-051, and ETBX-061), yeast-based vaccines (GI-6207 and GI-6301), haNK cells, aldoxorubicin, aspirin, avelumab, cyclophosphamide, nab-paclitaxel, and omega-3-acid ethyl esters will be repeated every 3 weeks. Concurrent SBRT will be given during the first 4 cycles. Radiation will be administered to no more than 5 feasible tumor sites using SBRT.

The neoadjuvant phase of treatment will be conducted in accordance with the following dosing regimen:

Daily: Aspirin (81 mg PO—discontinued 2 weeks prior to surgery); Omega-3-acid ethyl esters (by mouth [PO] twice a day [BID] [3×1 g capsules and 2×1 g capsules]).

Day 1, every 3 weeks: Cyclophosphamide (500 mg/m² PO).

Days 1 and 8, every 3 weeks: Nab-paclitaxel (100 mg/m² IV); Aldoxorubicin (65 mg/m²).

Day 5 (every 3 weeks for 3 doses then every 8 weeks thereafter): ETBX-011, ETBX-051, ETBX-061 (1×10¹¹ virus particles [VP]/vaccine/dose subcutaneously [SC]); GI-6207, GI-6301 (40 yeast units [YU]/vaccine/dose SC), 2 hours after administration of Ad5-based vaccines.

Day 8, every 3 weeks: Avelumab (10 mg/kg IV over 1 hour).

Days 8, 15, 22, 29: SBRT (not to exceed 8 Gy, exact dose to be determined by the radiation oncologist).

Days 9 and 16, every 3 weeks: ALT-803 (10 μg/kg SC 30 minutes prior to haNK infusion).

Days 9, 11, 16, and 18 every 3 weeks: haNK (2×10⁹ cells/dose IV).

Post-Operative Phase:

The duration of the post-operative phase will be 6 weeks following completion of the last treatment in the neoadjuvant phase and will include the following dosing regimen:

Day 1, weekly: Paclitaxel (100 mg IV)

Subjects will then enter the post-operative phase where they may remain for up to 6 weeks. Treatment will continue in the post-operative phase unless they experience unacceptable toxicity. The maximum time on treatment is 18 weeks in the neoadjuvant phase and 6 weeks in the post-operative phase.

Day 1, every 2 weeks for 8 weeks: Aldoxorubicin (65 mg/m²); Cyclophosphamide (600 mg/m² IV)

Followed by: Day 1, weekly for 10 weeks: Paclitaxel (80 mg/m²)

After 18 weeks, subjects will undergo CT or MRI to determine their current response status (ie, CR, PR, SD, or PD). Subjects will then undergo appropriate breast surgery and node dissection after which pCR will be evaluated.

Combination Immunotherapy in Subjects with Prostate Cancer

Therapeutic compositions and modalities used include various biological molecules and compositions as shown in Table 3 below.

TABLE 3

| | |
|---|---|
| ALT-803 | Recombinant human super agonist interleukin-15 (IL-15) complex, |
| | IL-15N72D: IL-15RαSu/IgG1 Fc complex (Altor Bioscience Corp., |
| | 2810 N Commerce Pkwy, Miramar, FL 33025) |
| ETBX-051 | Recombinant Adenovirus (Ad5 [E1-, E2b-]-Brachyury) that |
| | encodes Brachyury for expression of Brachyury in infected cells |
| ETBX-061 | Recombinant Adenovirus (Ad5 [E1-, E2b-]-MUC1) that encodes |
| | MUC1 for expression of MUC1 in infected cells |
| ETBX-071 | Recombinant Adenovirus (Ad5 [E1-, E2b-]-PSA) that encodes |
| | PSA for expression of PSA in infected cells |
| GI-4000 | Heat-killed S. cerevisiae yeast expressing RAS mutant proteins |
| GI-6301 | Heat-killed S. cerevisiae yeast expressing the human Brachyury |
| | (hBrachyury) oncoprotein |
| NK-92[CD16.158V, | NK92 derivative cells with high affinity CD16 variant and recombinant |
| ER IL-2] | intracellular expression of IL-2 (high-affinity activated Natural Killer |
| | cells, [haNK ™], NantKwest, 9920 Jefferson Blvd. Culver City, CA 90232) |
| Aldoxorubicin | Doxorubicin derivative ((6-maleimidocaproyl) hydrazone of doxorubicin). |
| Bevacizumab | Avastin (VEGF antibody) |
| 5-FU | 5-Fluorouracil |
| Avelumab | Bavencio (Fully human anti-PD-L1 IgG1 lambda monoclonal antibody) |
| Cyclophosphamide | 2-[bis(2-chloroethyl)amino]tetrahydro-2H-1,3,2-oxazaphosphorine 2-oxide |
| | monohydrate |
| Capecitabine | XELODA ® tablets, for oral use |
| Leucovorin | LEUCOVORIN Calcium for Injection, for IV or intramuscular [IM] use |
| Abraxane | Nab-paclitaxel (albumin bound paclitaxel) |
| Omega-3-acid ethyl | Lovaza (Omega-3-acid ethyl esters) |
| esters | |
| Radiation | Stereotactic Body Radiation Therapy (SBRT); 8 Gy maximum |
| | (exact dose to be determined by the radiation oncologist) |

Treatment will be administered in 2 phases, an induction and a maintenance phase, as described below. Subjects will continue induction treatment for up to 1 year or until they experience progressive disease (PD) or experience unacceptable toxicity (not correctable with dose reduction. Those who have a complete response (CR) in the induction phase will enter the maintenance phase. Subjects may remain on the maintenance phase for up to 1 year. Treatment will continue in the maintenance phase until the subject experiences PD or unacceptable toxicity. The maximum time on treatment, including both the induction and maintenance phases, is 2 years.

Tumor biopsies and exploratory tumor molecular profiling will be conducted at screening, at the end of the initial induction phase (8 weeks after the start of treatment), and during potential prolonged induction and maintenance phases (depending on response). Separate blood tubes will be collected every 4 weeks in the induction phase and every 8 weeks in the maintenance phase during routine blood draws for exploratory immunology and ctDNA/ctRNA analyses.

Tumors will be assessed at screening, and tumor response will be assessed every 8 weeks during the induction phase and every 12 weeks during the maintenance phase by computed tomography (CT), magnetic resonance imaging (MRI), or positron emission tomography-computed tomography (PET CT) of target and non-target lesions in accordance with Response Evaluation Criteria in Solid Tumors (RECIST) Version 1.1 and immune-related response criteria (irRC).

Prospective Tumor Molecular Profiling: Prospective tumor molecular profiling will be conducted to inform RAS mutational status and will be used to determine whether GI-4000 will be administered. All subjects will receive all other agents regardless of their tumor molecular profile. Prospective tumor molecular profiling will be performed on FFPE tumor tissue and whole blood (subject-matched normal comparator against the tumor tissue) collected at screening.

Subjects will receive GI-4000 if their tumor is positive for specific RAS mutations, as determined by whole genome sequencing. GI-4000 is 4 separate products from the GI-4000 series (GI-4014, GI-4015, GI-4016, and GI-4020); each of these expresses a combination of mutated RAS oncoproteins. The specific RAS mutation will determine which GI-4000 product will be used for treatment (GI-4014 for G12V, GI-4015 for G12C, GI-4016 for G12D, GI-4020 for G12R or Q61H, and GI-4014, GI-4015, or GI-4016 for Q61L or Q61R).

Induction Phase:

The induction phase will include repeated 2 week cycles. The treatment regimen of aldoxorubicin, ALT-803, Ad5 based vaccines (ETBX-051, ETBX-061, and ETBX-071), yeast-based vaccines (GI-4000 and GI-6301), haNK cells, avelumab, bevacizumab, cyclophosphamide, 5 FU/leucovorin, nab-paclitaxel, and omega-3-acid ethyl esters will be repeated every 2 weeks. Concurrent SBRT will be given during the first four 2-week cycles. Radiation will be administered to no more than 5 feasible tumor sites using SBRT. The induction phase will be conducted in accordance with the following dosing regimen:

Daily: Omega-3-acid ethyl esters (by mouth [PO] twice a day [BID] [3×1 g capsules and 2×1 g capsules]).

Day 1, every 2 weeks: Bevacizumab (5 mg/kg IV).

Days 1-5 and 8-12, every 2 weeks: Cyclophosphamide (50 mg PO BID).

Days 1, 3, 5, 8, 10 and 12, every 2 weeks: 5-FU (400 mg/m² as a continuous IV infusion over 24 hours); Leucovorin (20 mg/m² IV bolus).

Day 1 and 8, every 2 weeks: Nab-paclitaxel (100 mg IV); Aldoxorubicin (20 mg/m²).

Day 5, 19, 33 (every 2 weeks for 3 doses then every 8 weeks thereafter): ETBX-051, ETBX-061, ETBX-071 (5×10¹¹ virus particles [VP]/vaccine/dose subcutaneously [SC]); GI-4000, GI-6301 (40 yeast units [YU]/vaccine/dose SC), 2 hours after administration of Ad5-based vaccines.

Prospective tumor molecular profiling will determine whether GI-4000 will be administered as described above.

Day 8, every 2 weeks: Avelumab (10 mg/kg IV over 1 hour).

Day 8, 22, 36, 50 (every 2 weeks for 4 doses): SBRT (not to exceed 8 Gy, exact dose to be determined by the radiation oncologist).

Day 9, every 2 weeks: ALT-803 (10 μg/kg SC 30 minutes prior to haNK infusion)

Day 9 and 11, every 2 weeks: haNK (2×10⁹ cells/dose IV).

Maintenance Phase:

The duration of the maintenance phase will be up to 1 year following completion of the last treatment in the induction phase. The maintenance phase will include repeated 2-week cycles. The treatment regimen of ALT-803, Ad5 based vaccines (ETBX 051, ETBX 061, and ETBX-071), yeast-based vaccines (GI-4000 and GI-6301), haNK cells, avelumab, bevacizumab, capecitabine, cyclophosphamide, nab-paclitaxel, and omega-3-acid ethyl esters will be repeated every 2 weeks. The maintenance phase will be conducted in accordance with the following dosing regimen:

Daily: Omega-3-acid ethyl esters (PO BID [3×1 g capsules and 2×1 g capsules]).

Day 1, every 2 weeks: Bevacizumab (5 mg/kg IV); Nab-paclitaxel (100 mg IV); Avelumab (10 mg/kg IV over 1 hour).

Days 1-5 and 8-12, a every 2 weeks: Cyclophosphamide (50 mg PO BID); Capecitabine (650 mg/p (PA BD).

Day 2, every 2 weeks: ALT-803 (10 g g/kg SC 30 minutes prior to haNK infusion); haNK ((2×10⁹ cells/dose IV).

Day 5, every 8 weeks thereafter: ETBX-051, ETBX-061, ETBX-071 (5×10¹¹ VP/vaccine/dose SC); GI-4000, GI-6301 (40YU/dose SC), 2 hours after administration of Ad5-based vaccines.

Prospective tumor molecular profiling will determine whether GJ-4000 will be administered, as described above.

Combination Immunotherapy in Subjects with Recurrent or Metastatic Sarcoma

Therapeutic compositions and modalities used include various biological molecules and compositions as shown in Table 4 below.

TABLE 4

| | |
|---|---|
| ALT-803 | Recombinant human super agonist interleukin-15 (IL-15) complex, |
| | IL-15N72D: IL-15RαSu/IgG1 Fc complex (Altor Bioscience Corp., |
| | 2810 N Commerce Pkwy, Miramar, FL 33025) |
| ETBX-051 | Recombinant Adenovirus (Ad5 [E1-, E2b-]-Brachyury) that encodes |
| | Brachyury for expression of Brachyury in infected cells |
| ETBX-061 | Recombinant Adenovirus (Ad5 [E1-, E2b-]-HER2) that encodes |
| | HER2 for expression of HER2 in infected cells |
| ETBX-021 | Recombinant Adenovirus (Ad5 [E1-, E2b-]-PSA) that encodes PSA for |
| | expression of PSA in infected cells |
| GI-4000 | Heat-killed S. cerevisiae yeast expressing RAS mutant proteins |
| GI-6301 | Heat-killed S. cerevisiae yeast expressing the human Brachyury |
| | (hBrachyury) oncoprotein |
| NK-92[CD16.158V, | NK92 derivative cells with high affinity CD16 variant and recombinant |
| ER IL-2] | intracellular expression of IL-2 (high-affinity activated Natural Killer |
| | cells, [haNK ™], NantKwest, 9920 Jefferson Blvd. Culver City, CA 90232) |
| Aldoxorubicin | Doxorubicin derivative ((6-maleimidocaproyl) hydrazone of doxorubicin). |
| Bevacizumab | Avastin (VEGF antibody) |
| Trabectedin | Yondelis ® for injection, for IV use |
| Avelumab | Bavencio (Fully human anti-PD-L1 IgG1 lambda monoclonal antibody) |
| Cyclophosphamide | 2-[bis(2-chloroethyl)amino]tetrahydro-2H-1,3,2-oxazaphosphorine |
| | 2-oxide monohydrate |
| Abraxane | Nab-paclitaxel (albumin bound paclitaxel) |
| Omega-3-acid ethyl | Lovaza (Omega-3-acid ethyl esters) |
| esters | |
| Radiation | Stereotactic Body Radiation Therapy (SBRT); 8 Gy maximum |
| | (exact dose to be determined by the radiation oncologist) |

Treatment will be administered in 2 phases, an induction and a maintenance phase, as described below. Subjects will continue induction treatment for up to 1 year. Treatment will be discontinued if the subject experiences progressive disease (PD) or unacceptable toxicity (not corrected with dose reduction). Those who have a complete response (CR) in the induction phase will enter the maintenance phase. Subjects may remain on the maintenance phase for up to 1 year. Treatment will continue in the maintenance phase until the subject experiences PD or unacceptable toxicity (not corrected with dose reduction). The maximum time on treatment, including both the induction and maintenance phases, is to 2 years.

Tumor biopsies and exploratory tumor molecular profiling will be conducted at screening, at the end of the initial induction phase (8 weeks after the start of treatment), during a potential prolonged induction phase (depending on response), and during a maintenance phase. Separate blood tubes will be collected every 4 weeks in the induction phase and every 8 weeks in the maintenance phase during routine blood draws for exploratory immunology and ctDNA/ctRNA analyses.

Tumors will be assessed at screening, and tumor response will be assessed every 8 weeks during the induction phase and every 12 weeks during the maintenance phase by computed tomography (CT), magnetic resonance imaging (MRI), or positron emission tomography (PET)-CT of target and non-target lesions in accordance with Response Evaluation Criteria in Solid Tumors (RECIST) Version 1.1 and immune-related response criteria (irRC).

Prospective Tumor Molecular Profiling: Prospective tumor molecular profiling will be conducted to inform HER2 expression and RAS mutational status, and will be used to determine whether ETBX-021 and/or GI-4000 will be administered. All subjects will receive all other agents regardless of their tumor molecular profile. Prospective tumor molecular profiling will be performed on FFPE tumor tissue and whole blood (subject-matched normal comparator against the tumor tissue) collected at screening. Subjects will receive ETBX-021 if their tumor overexpresses HER2 (≥750 attomole/μg of tumor tissue, as determined by quantitative proteomics with mass spectrometry). Subjects will receive GI-4000 if their tumor is positive for specific RAS mutations, as determined by whole genome sequencing. GI-4000 is 4 separate products from the GI-4000 series (GI-4014, GI-4015, GI-4016, and GI-4020); each of these expresses a combination of mutated RAS oncoproteins. The specific RAS mutation will determine which GI-4000 product will be used for treatment (GI-4014 for G12V, GI-4015 for G12C, GI-4016 for G12D, GI-4020 for G12R or Q61H, and GI-4014, GI-4015, or GI-4016 for Q61L or Q61R).

Induction Phase:

The induction phase will include repeated 2-week cycles for a maximum treatment period of 1 year. The treatment regimen of aldoxorubicin, ALT-803, avelumab, bevacizumab, cyclophosphamide, Ad5-based vaccines (ETBX-021, ETBX-051, and ETBX-061), yeast-based vaccines (GI-4000 and GI-6301), haNK cells, nab-paclitaxel, omega-3-acid ethyl esters, SBRT, and trabectedin will be repeated every 2 weeks. Concurrent SBRT will be given during the first four 2-week cycles. Radiation using SBRT will be administered to no more than 3 feasible tumor sites for the first 3 subjects and to no more than 5 feasible tumor sites for subsequently enrolled subjects. The induction phase will be conducted in accordance with the following dosing regimen:

Daily: Omega-3-acid ethyl esters (by mouth [PO] BID [3×1 g capsules and 2×1 g capsules]).

Day 1, every 2 weeks: Bevacizumab (5 mg/kg IV).

Days 1-5 and 8-12, every 2 weeks: Cyclophosphamide (50 mg PO twice a day [BID]).

Day 1 and 8, every 2 weeks: Aldoxorubicin (20 mg/m² IV); Nab-paclitaxel (100 mg IV); Trabectedin (0.2 mg/m² IV).

Day 5, 19, 33 (every 2 weeks for 3 doses then every 8 weeks thereafter): ETBX-021, ETBX-051, ETBX-061 (1×10¹¹ virus particles [VP]/vaccine/dose subcutaneously [SC]); GI-4000, GI-6301 (40 yeast units [YU]/vaccine/dose SC), 2 hours after administration of the Ad5-based vaccines. Prospective tumor molecular profiling will determine whether ETBX-021 and/or GI-4000 will be administered, as described above.

Day 8, every 2 weeks: Avelumab (10 mg/kg IV over 1 hour).

Day 8, 22, 36, 50 (every 2 weeks for 4 doses): SBRT (a maximum of 6 Gy or 8 Gy). SBRT will be administered to a maximum of 5 target lesions at doses of up to 8 Gy. For all subjects, the exact dose of radiation to be administered will be determined by the radiation oncologist.

Day 9, every 2 weeks: ALT-803 (10 μg/kg SC 30 minutes prior to haNK infusion).

Day 9 and 11, every 2 weeks: haNK (2×10⁹ cells/dose IV).

Maintenance Phase:

The duration of the maintenance phase will be up to 1 year following completion of the last treatment in the induction phase. The maintenance phase will include repeated 2-week cycles. The treatment regimen of ALT-803, avelumab, bevacizumab, cyclophosphamide, Ad5-based vaccines (ETBX-021, ETBX-051, and ETBX-061), yeast-based vaccines (GI-4000 and GI-6301), haNK cells, nab-paclitaxel, omega-3-acid ethyl esters, and trabectedin will be repeated every 2 weeks. The maintenance phase will be conducted in accordance with the following dosing regimen:

Daily: Omega-3-acid ethyl esters (PO BID [3×1 g capsules and 2×1 g capsules]).

Day 1, every 2 weeks: Avelumab (10 mg/kg IV over 1 hour); Bevacizumab (5 mg/kg IV); Nab-paclitaxel (100 mg IV); Trabectedin (0.2 mg/m² IV).

Days 1-5 and 8-12, every 2 weeks: Cyclophosphamide (50 mg PO BID).

Day 2, every 2 weeks: ALT-803 (10 μg/kg SC) (30 minutes prior to haNK infusion); haNK (2×10⁹ cells/dose IV).

Day 5, every 8 weeks thereafter: ETBX-021, ETBX-051, ETBX-061 (1×10¹¹ VP/vaccine/dose SC); GI-4000, GI-6301 (40 YU/vaccine/dose SC), 2 hours after administration of the Ad5 based vaccines. Prospective tumor molecular profiling will determine whether ETBX-021 and/or GI-4000 will be administered, as described above.

Combination Immunotherapy in Subjects with Advanced Chordoma

Therapeutic compositions and modalities used include various biological molecules and compositions as shown in Table 5 below.

TABLE 5

| | |
|---|---|
| ALT-803 | Recombinant human super agonist interleukin-15 (IL-15) complex, |
| | IL-15N72D: IL-15RαSu/IgG1 Fc complex (Altor Bioscience Corp., |
| | 2810 N Commerce Pkwy, Miramar, FL 33025) |
| ETBX-051 | Recombinant Adenovirus (Ad5 [E1-, E2b-]-Brachyury) that encodes |
| | Brachyury for expression of Brachyury in infected cells |
| GI-6301 | Heat-killed S. cerevisiae yeast expressing the human Brachyury |
| | (hBrachyury) oncoprotein |
| NK-92[CD16.158V, | NK92 derivative cells with high affinity CD16 variant and recombinant |
| ER IL-2] | intracellular expression of IL-2 (high-affinity activated Natural Killer |
| | cells, [haNK ™], NantKwest, 9920 Jefferson Blvd. Culver City, CA 90232) |
| Aldoxorubicin | Doxorubicin derivative ((6-maleimidocaproyl) hydrazone of doxorubicin). |
| Bevacizumab | Avastin (VEGF antibody) |
| Cetuximab | ERBITUX ® injection, for IV infusion |
| Trabectedin | Yondelis ® for injection, for IV use |
| Avelumab | Bavencio (Fully human anti-PD-L1 IgG1 lambda monoclonal antibody) |
| Cyclophosphamide | 2-[bis(2-chloroethyl)amino]tetrahydro-2H-1,3,2-oxazaphosphorine |
| | 2-oxide monohydrate |
| Abraxane | Nab-paclitaxel (albumin bound paclitaxel) |
| Omega-3-acid ethyl | Lovaza (Omega-3-acid ethyl esters) |
| esters | |
| Radiation | Stereotactic Body Radiation Therapy (SBRT); 8 Gy maximum |
| | (exact dose to be determined by the radiation oncologist) |

Treatment will be administered in 2 phases, an induction and a maintenance phase, as described below. Subjects will continue induction treatment for up to 1 year or until they experience progressive disease (PD) or unacceptable toxicity (not correctable with dose reduction). Those who have a complete response (CR) in the induction phase will enter the maintenance phase. Subjects may remain in the maintenance phase for up to 1 year. Treatment will continue in the maintenance phase until the subject experiences PD or unacceptable toxicity (not correctable with dose reduction). The maximum time on treatment, including both the induction and maintenance phases, is 2 years.

Tumor biopsies and exploratory tumor molecular profiling will be conducted at screening, at the end of the initial induction phase (8 weeks after the start of treatment), and during potential prolonged induction and maintenance phases (depending on response). Separate blood tubes will be collected every 4 weeks in the induction phase and every 8 weeks in the maintenance phase during routine blood draws for exploratory immunology and ctDNA/ctRNA analyses. Tumors will be assessed at screening, and tumor response will be assessed every 8 weeks during the induction phase and every 12 weeks during the maintenance phase by computed tomography (CT), magnetic resonance imaging (MRI), or positron emission tomography (PET)-CT of target and non-target lesions in accordance with Response Evaluation Criteria in Solid Tumors (RECIST) Version 1.1 and immune-related response criteria (irRC).

Induction Phase:

The induction phase will consist of repeated 2-week cycles for a maximum treatment period of 1 year. The treatment regimen consists of ALT-803, avelumab, bevacizumab, cetuximab, cyclophosphamide, aldoxorubicin, ETBX-051, GI-6301, haNK cells, nab-paclitaxel, omega-3-acid ethyl esters, trabectedin, and radiation therapy. Concurrent SBRT will be given during the first four 2-week cycles. Radiation will be administered to no more than 5 feasible tumor sites using SBRT. The induction phase will be conducted in accordance with the following dosing regimen:

Daily: Omega-3-acid ethyl esters (by mouth [PO] twice a day [BID] [3×1 g capsules and 2×1 g capsules]).

Day 1, every 2 weeks: Bevacizumab (5 mg/kg IV).

Days 1-5 and 8-12, every 2 weeks: Cyclophosphamide (50 mg PO BID).

Day 1 and 8, every 2 weeks: Nab-paclitaxel (75 mg IV); Aldoxorubicin (25 mg/m² IV); Trabectedin (0.2 mg/m² IV).

Day 5, 19, 33 (every 2 weeks for 3 doses then every 8 weeks thereafter): ETBX-051 (5×10¹¹ virus particles [VP]/vaccine/dose subcutaneously [SC]); GI-6301 (40 yeast units [YU]/vaccine/dose SC), 2 hours after administration of ETBX-051.

Day 8, every week: Cetuximab (250 mg IV).

Day 8, every 2 weeks: Avelumab (10 mg/kg IV over 1 hour).

Day 8, 22, 36, 50 (every 2 weeks for 4 doses): SBRT (not to exceed 8 Gy, exact dose to be determined by the radiation oncologist).

Day 9, every 2 weeks: ALT-803 (10 μg/kg SC 30 minutes prior to haNK infusion).

Day 9 and 11, every 2 weeks: haNK (2×10⁹ cells/dose IV).

Maintenance Phase:

The duration of the maintenance phase will be up to 1 year following completion of the last treatment in the induction phase. The maintenance phase will consist of repeated 2-week cycles. The treatment regimen consists of ALT-803, avelumab, bevacizumab, cetuximab, cyclophosphamide, ETBX-051, GI-6301, haNK cells, nab-paclitaxel, omega-3-acid ethyl esters, and trabectedin. The maintenance phase will be conducted in accordance with the following dosing regimen:

Daily: Omega-3-acid ethyl esters (PO BID [3×1 g capsules and 2×1 g capsules]).

Day 1, every 2 weeks: Bevacizumab (5 mg/kg IV); Nab-paclitaxel (75 mg IV); Avelumab (10 mg/kg IV over 1 hour); Cetuximab (250 mg IV); Trabectedin (0.2 mg/m² IV).

Days 1-5 and 8-12, every 2 weeks: Cyclophosphamide (50 mg PO BID).

Day 2, every 2 weeks: ALT-803 (10 μg/kg SC) (30 minutes prior to haNK infusion); haNK (2×10⁹ cells/dose IV).

Day 5, every 8 weeks thereafter: ETBX-051 (5×10¹¹ VP/vaccine/dose SC); GI-6301 (40 YU/vaccine/dose SC), 2 hours after administration of ETBX-051.

Combination Immunotherapy in Subjects with Metastatic Pancreatic Carcinoma

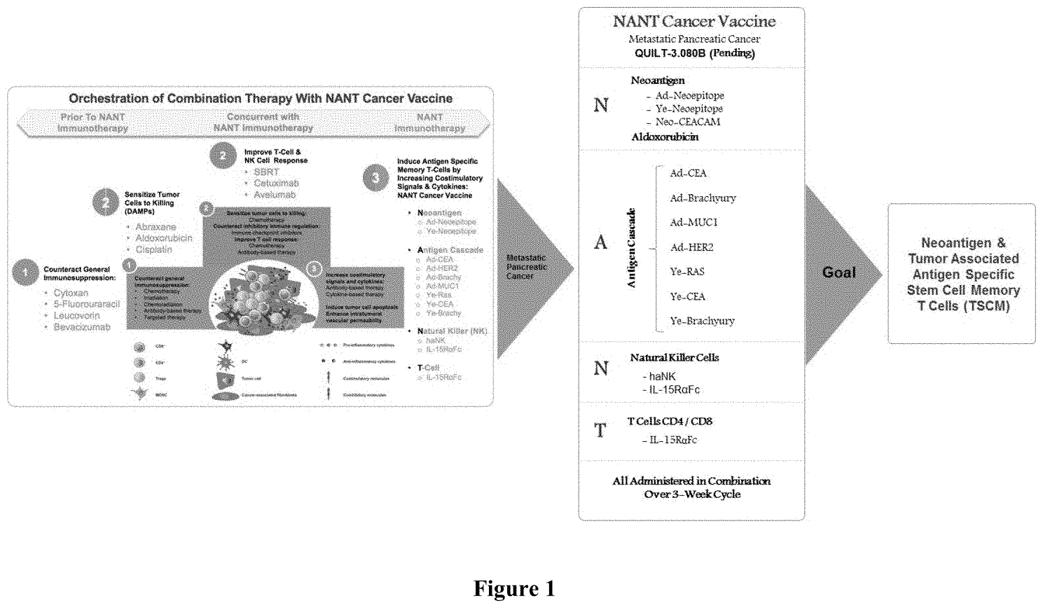

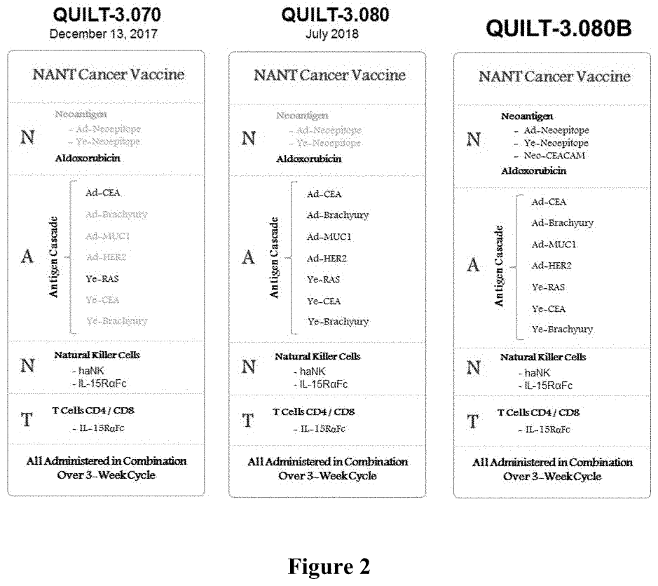

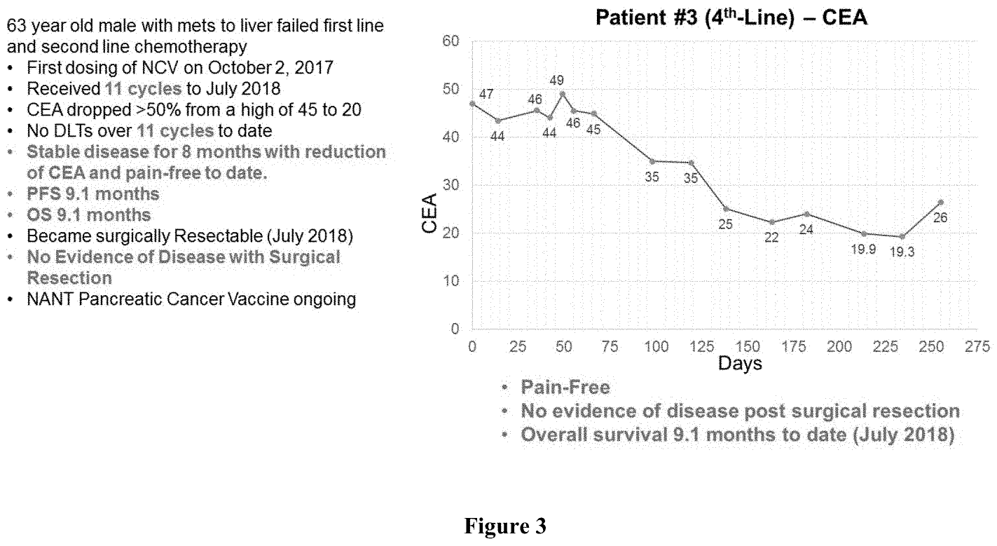

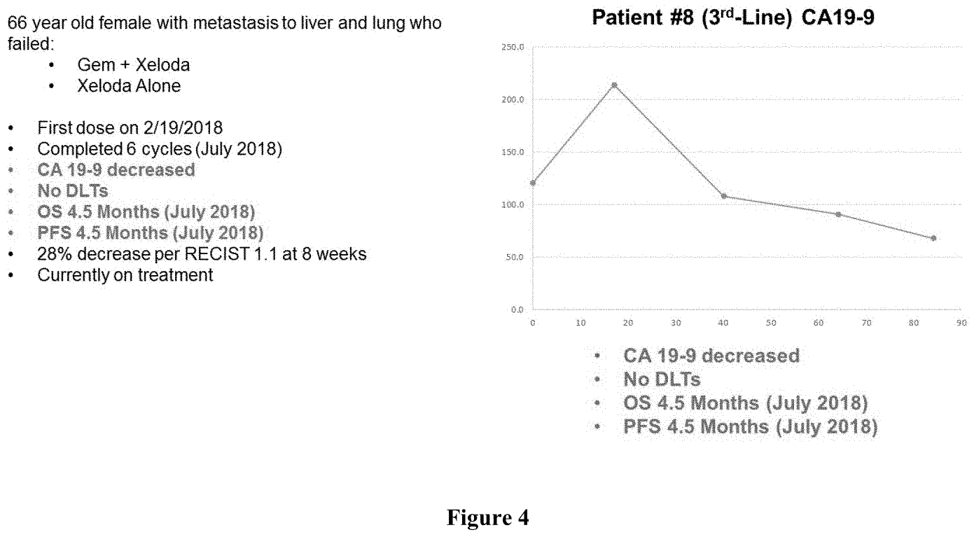

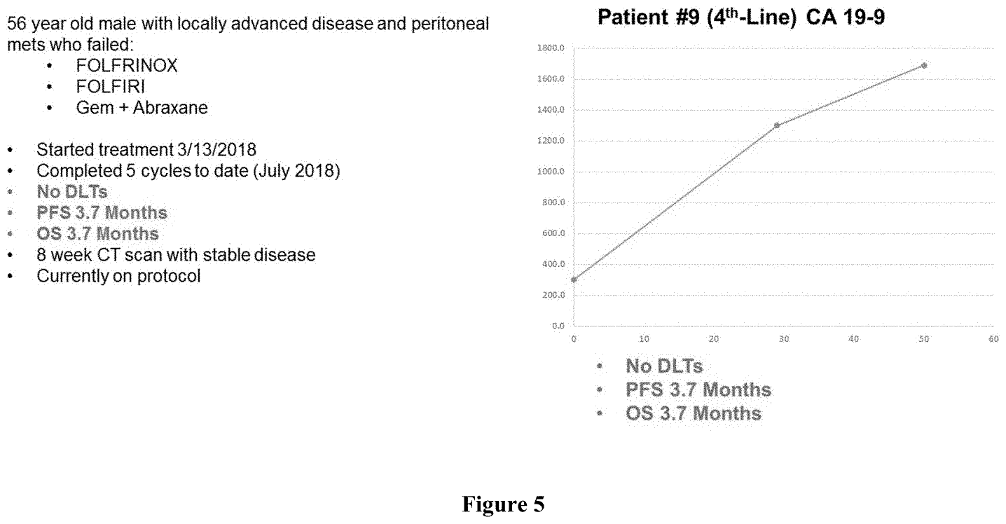

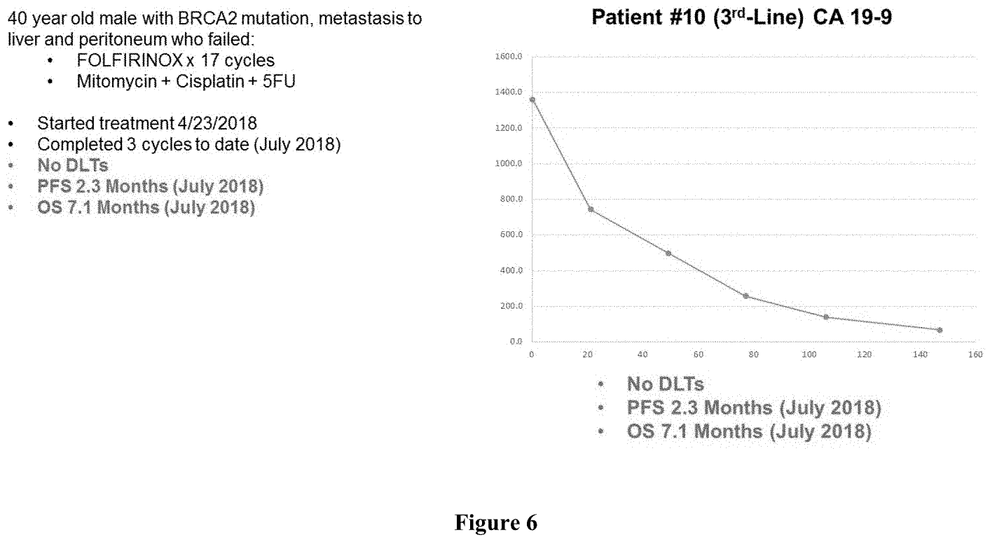

Subjects with metastatic pancreatic cancer were treated in a manner as recorded in US Clinical trials with Identifiers NCT03329248 and NCT03387098. FIG. 1 schematically illustrates the treatment strategy and modalities in which immune suppression in the tumor microenvironment is first reduced (here: using aldoxorubicin), and in which immune therapy is administered (here: using recombinant adenovirus/yeast vaccine, plus modified natural killer cells) to trigger an antigen cascade and stimulate formation of memory T cells (and particularly T_{SCM} cells). FIG. 2 shown in more detail the modalities used in the treatment of pancreatic cancer (3.070/3.080/3.080B). As can be seen from FIG. 2, aldoxorubicin is used to reduce/eliminate immune suppression in the tumor microenvironment, which is followed by administration of an recombinant adenovirus (encoding CEA (3.070), and additionally encoding further tumor associated antigens (3.080)) and recombinant yeast (encoding RAS (3.070), and additionally encoding further tumor associated antigens (3.080)). Immune therapy also included use of modified NK cells (here: NK cells with high affinity variant of CD16, active in hypoxic tumor microenvironment). Further treatment support was given using ALT-803 (IL-15 chimeric protein, Altor Bioscience). As can be seen from the results in FIGS. 3-6 for selected patients, treatment response was significant.

Combination Immunotherapy in Subjects with Metastatic Triple Negative Breast Cancer

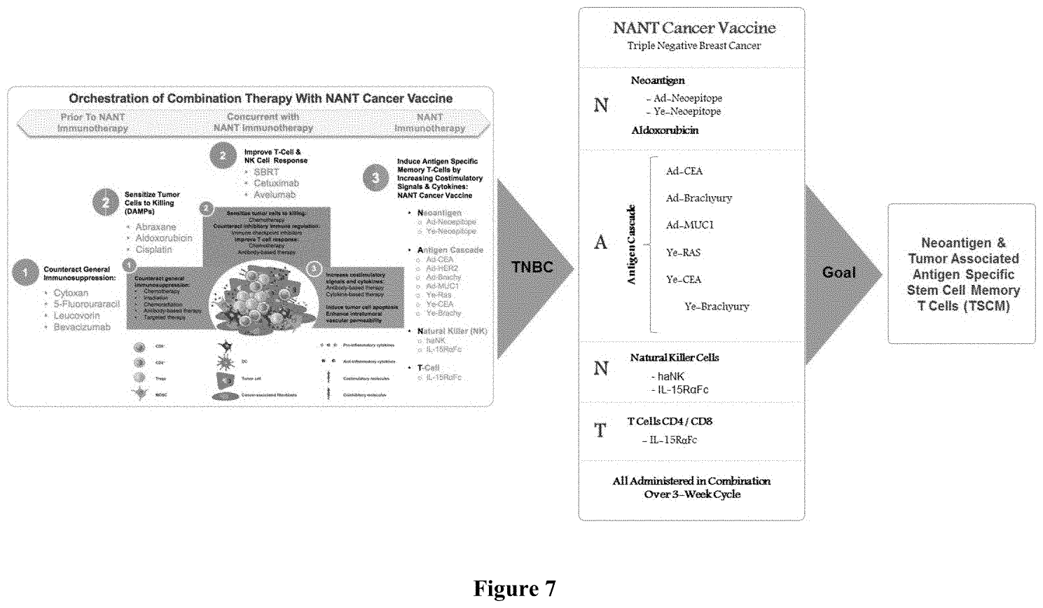

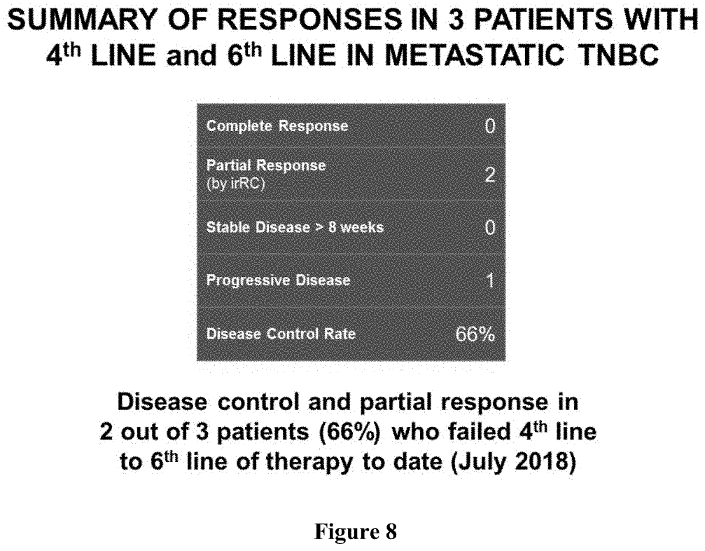

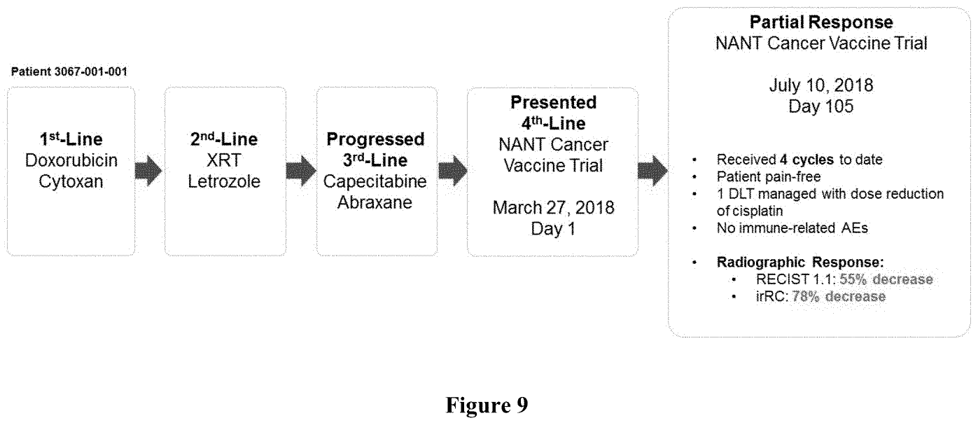

Subjects with metastatic pancreatic cancer were treated in a manner as recorded in US Clinical trial with Identifier NCT03554109. FIG. 7 schematically illustrates treatment strategy and modalities in which immune suppression in the tumor microenvironment is first reduced (here: using aldoxorubicin), and in which immune therapy is then administered (here: using recombinant adenovirus/yeast vaccine, plus modified natural killer cells) to trigger an antigen cascade and stimulate formation of memory T cells (and particularly T_{SCM} cells). FIG. 8 depicts exemplary results for such treatment strategy, and FIG. 9 provides an exemplary patient result.

Combination Immunotherapy in Subjects with Metastatic Squamous Cell Carinoma

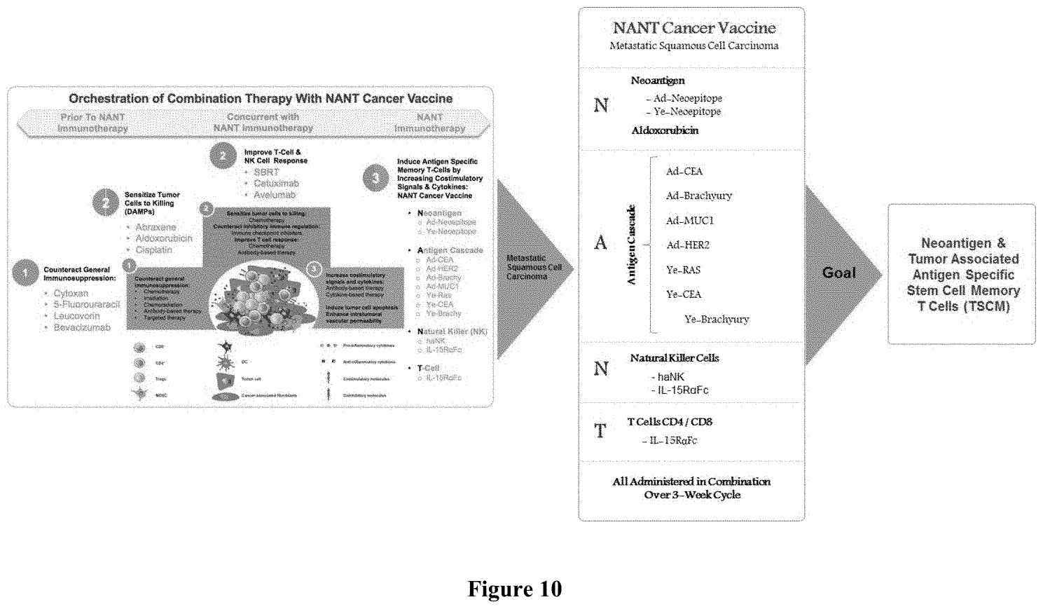

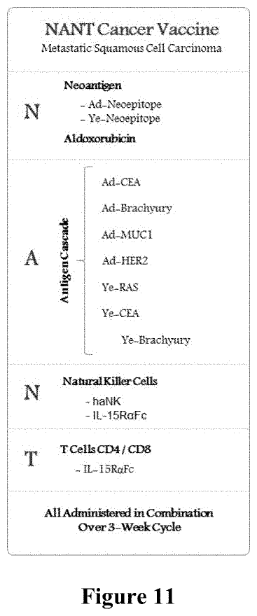

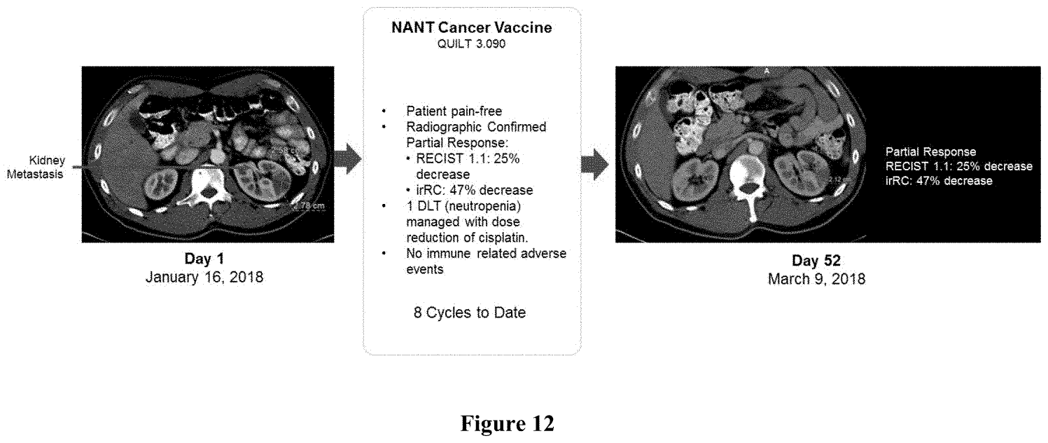

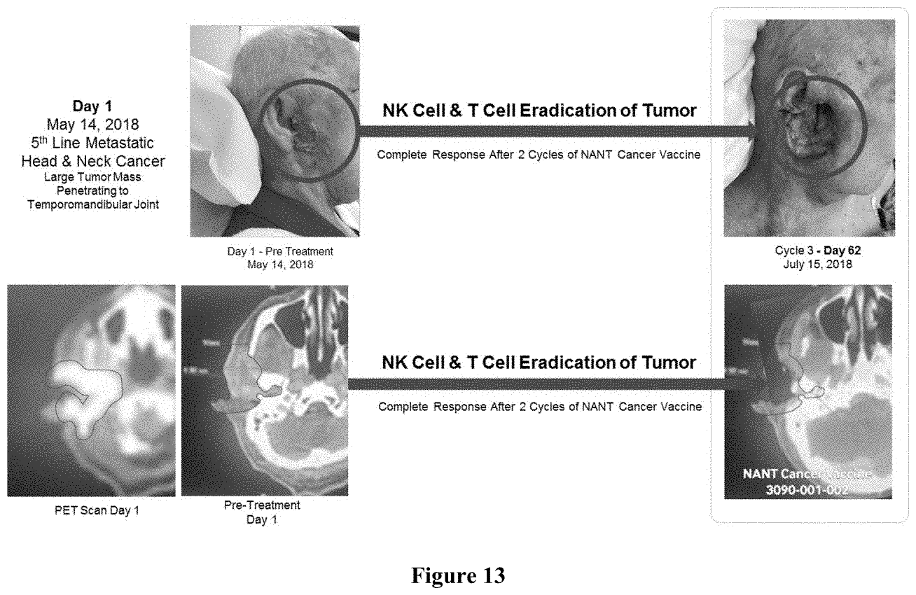

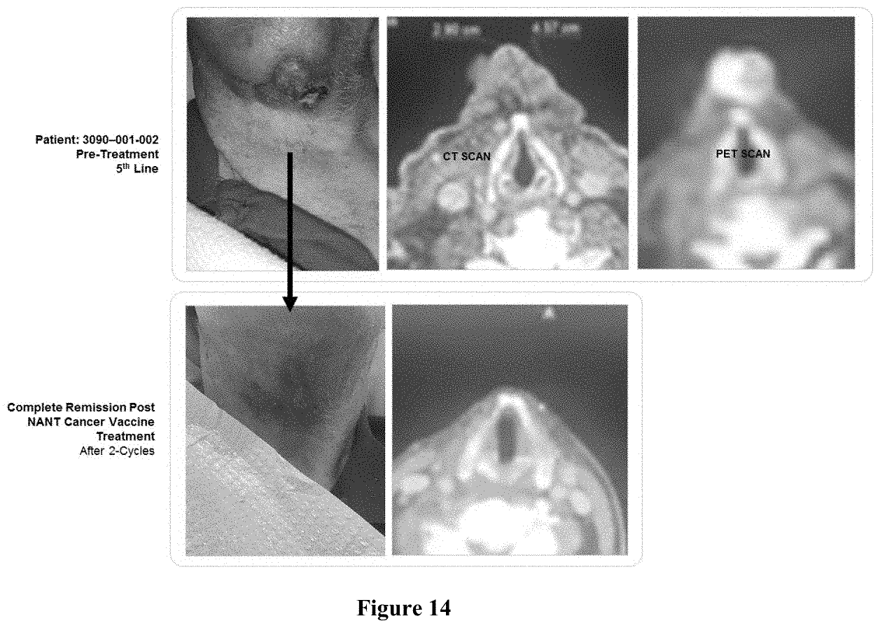

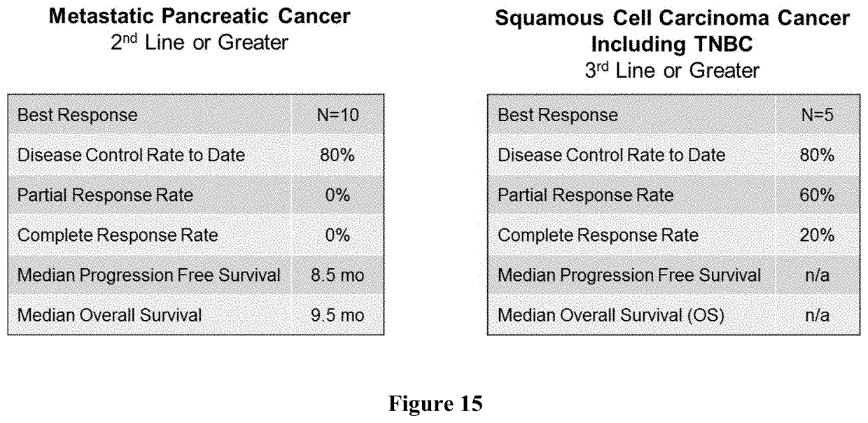

Subjects with metastatic pancreatic cancer were treated in a manner as recorded in US Clinical trial with Identifier NCT03387111. FIG. 10 schematically illustrates treatment strategy and modalities in which immune suppression in the tumor microenvironment is first reduced (here: using aldoxorubicin), and in which immune therapy is then administered (here: using recombinant adenovirus/yeast vaccine, plus modified natural killer cells) to trigger an antigen cascade and stimulate formation of memory T cells (and particularly T_{SCM} cells). FIG. 11 depicts exemplary treatment modalities as noted above, and FIGS. 12-14 depict exemplary results for such treatment strategy. FIG. 15 shows exemplary summaries of results for various cancers using treatment strategies presented herein.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. The recitation of ranges of values herein is merely intended to serve as a shorthand method of referring individually to each separate value falling within the range. Unless otherwise indicated herein, each individual value with a range is incorporated into the specification as if it were individually recited herein.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

1. A method of treating a tumor, comprising:
treating tumor cells within an acidic and hypoxic tumor microenvironment with at least a first pharmaceutical composition that reduces immune suppression in the tumor microenvironment to thereby revert an escape phase of the tumor cells; and
treating the tumor cells with an immune therapeutic composition that comprises a vaccine component and a cell-based component to thereby induce an elimination phase of the tumor cells.

2. The method of claim 1 further comprising a step of maintaining an equilibrium phase of the tumor cells by administering at least a second pharmaceutical composition that biases an immune response towards a T_{H}1 response.

3. The method of claim 1 wherein the first pharmaceutical composition comprises a drug that binds to a thiol group of an albumin or a drug that is bound to an albumin, wherein the albumin is optionally a nanoparticulate albumin.

4. The method of claim 3 wherein drug that binds to the thiol group is aldoxorubicin.

5. The method of claim 3 further comprising an antibody or fragment thereof bound to the albumin.

6. The method of claim 3 wherein the drug is Bendamustine, Bortezomib, Cabazitaxel, Chlorambucil, Cisplatin, Cyclophosphamide, Dasatinib, Docetaxel, Doxorubicin, Epirubicin, Erlotinib, Etoposide, Everolimus, Gefitinib, Idarubicin, Hydroxyurea, Imatinib, Lapatinib, Melphalan, Mitoxantrone, Nilotinib, Oxiplatin, Paclitaxel, Pazopanib, Pemetrexed, Rapamycin, Romidepsin, Sorafenib, Vemurafenib, Sunitinib, Teniposide, Vinblastine, Vinorelbine, or Vincristine.

7. The method of claim 1 wherein the first pharmaceutical composition comprises a drug that inhibits at least one of a T-reg cell, a myeloid derived suppressor cell, and a M2 macrophage.

8. The method of claim 7 wherein the drug is selected from the group consisting of cisplatin, gemcitabine, 5-fluorouracil, cyclophosphamide, aldoxorubicin, doxorubicin, temozolomide, docetaxel, paclitaxel, trabectedin, and RP-182.

9. The method of claim 1 wherein the first pharmaceutical composition comprises a vascular permeability enhancer.

10. The method of claim 9 wherein the first vascular permeability enhancer comprises at least a portion of IL2.

11. The method of claim 1 wherein the vaccine component comprises a recombinant bacterial vaccine, a recombinant viral vaccine, or a recombinant yeast vaccine.

12. The method of claim 11 wherein the recombinant bacterial vaccine, the recombinant viral vaccine, or the recombinant yeast vaccine is genetically engineered to express at least one of a cancer associated antigen, a cancer specific antigen, and a patient- and tumor-specific neoepitope.

13. The method of claim 12 wherein the cancer associated antigen is selected from the group consisting of MUC1, CEA, HER2, Brachyury, and an oncogenic Ras mutant protein.

14. The method of claim 1 wherein the cell-based component comprises a natural killer cell.

15. The method of claim 14 wherein the natural killer cell is an aNK cell, a haNK cell, or a taNK cell.

16. The method of claim 1 further comprising a step of administering an immune stimulatory cytokine.

17. The method of claim 16 wherein the immune stimulatory cytokine is selected from the group consisting of IL-2, IL-7, IL-15, IL-17, IL-21, and an IL-15 superagonist.

18. The method of claim 1 further comprising a step of administering a checkpoint inhibitor.

19. The method of claim 18 wherein the checkpoint inhibitor is a PD-1 inhibitor or a CTLA4 inhibitor.

20. The method of claim 1 further comprising a step of administering low dose radiation to the tumor.

21-52. (canceled)
